(19) United States
(12) Patent Application Publication
(10) Pub. No.: US 2022/0378406 A1
(43) Pub. Date: Dec. 1, 2022

(54) CLOSURE DEVICES AND METHODS FOR SEALING BIOLOGIC TISSUE MEMBRANES

(71) Applicant: Deinde Medical Corp., Miramar, FL (US)

(72) Inventor: Robert C. Farnan, Fort Lauderdale, FL (US)

(21) Appl. No.: 17/883,118

(22) Filed: Aug. 8, 2022

Related U.S. Application Data
(63) Continuation of application No. PCT/US2021/016903, filed on Feb. 5, 2021.
(60) Provisional application No. 62/975,988, filed on Feb. 13, 2020.

(51) Int. Cl. A61B 17/00; A61B 90/00; A61B 17/34

(52) U.S. Cl. CPC A61B 17/0057; A61B 90/39; A61B 17/3468; A61B 2017/00623; A61B 2017/00601; A61B 2017/00743; A61B 2017/00592; A61B 2017/00681

(57) ABSTRACT

An implantable closure device for sealing an opening, such as a puncture, through a biologic tissue membrane, such as the meninges, against leakage of biological fluid. The closure device includes a fluid sealing plug configured to be positioned at least partially within the opening through the biologic tissue membrane. A retainer is configured to secure the fluid sealing plug at least partially within the opening. The retainer includes a proximal retainer portion configured to be disposed proximally on the biologic tissue membrane, a distal retainer portion configured to be disposed distally on the biologic tissue membrane, and a central retainer portion connecting the proximal retainer portion and the distal retainer portion. The central retainer portion is configured to extend through the opening. The fluid sealing plug is coupled to the central retainer portion.

### CROSS REFERENCE TO RELATED APPLICATION

This application is a continuation of PCT Application Serial No. PCT/US2021/016903, filed Feb. 5, 2021, which claims priority to U.S. Provisional Patent Application Ser. No. 62/975,988, filed Feb. 13, 2020, the disclosures of which are incorporated by reference herein in their entirety.

### TECHNICAL FIELD

The present invention generally relates to implantable tissue closure devices and, more particularly, to implantable closure devices for sealing punctures or other openings through biologic tissue membranes, such as the meninges, against leakage of biological fluids, such as cerebrospinal fluid, and related methods.

### BACKGROUND

The present disclosure contemplates that the meninges are protective biologic tissue membranes around the brain and spinal cord. The meninges contain the cerebrospinal fluid and generally form a conduit that surrounds the spinal cord and the cerebral ventricles. In some medical or surgical procedures, a needle or other instrument may be used to puncture through the skin, soft tissue, and the meninges, such as to gain access to the cerebrospinal fluid. When the instrument is removed, the hole or puncture may not seal spontaneously, such as due to the inelastic properties of the meninges. If the puncture does not promptly seal, the cerebrospinal fluid may leak into the adjacent soft tissue, which may not be clinically desirable. In other medical situations an opening may exist through tissue, and may be the result of an opening or some other natural or surgically formed tissue opening, or an injury, etc.

Accordingly, and despite the various advances already made in this field, there is a need for further improvements related to implantable tissue closure devices and, more particularly, to implantable closure devices for sealing openings, such as punctures through biologic tissue membranes, such as the meninges, and related methods for sealing openings.

### SUMMARY

Generally, an implantable closure device for sealing an opening through a biologic tissue membrane against leakage of biological fluid is provided. The closure device includes a fluid sealing plug configured to be positioned at least partially within the opening through a biologic tissue membrane. A retainer is configured to secure the fluid sealing plug at least partially within the opening. The retainer includes a proximal retainer portion configured to be disposed proximally on the biologic tissue membrane, a distal retainer portion configured to be disposed distally on the biologic tissue membrane, and a central retainer portion connecting the proximal retainer portion and the distal retainer portion. The central retainer portion is configured to extend through the opening. The fluid sealing plug is coupled to the central retainer portion.

The device may have various optional or additional features. The fluid sealing plug may be coupled to the retainer between the proximal retainer portion and the distal retainer portion. A generally radial outer surface of the fluid sealing plug may be configured to engage a generally radial inner surface of the opening to provide an at least partially sealed interface between the generally radial outer surface of the fluid sealing plug and the generally radial inner surface of the opening. The fluid sealing plug may be formed of a porous polymer material, such as expanded polytetrafluoroethylene or other materials. The retainer may be reconfigurable between a low-profile delivery configuration and an implanted configuration. In the implanted configuration, a diameter of the proximal retainer portion may be substantially greater than a diameter of the fluid sealing plug. For example, in the implanted configuration, the diameter of the proximal retainer portion may be about 1.25 to about 3.0 times of at least one of a diameter of the opening and a diameter of the fluid sealing plug. In the implanted configuration, a diameter of the distal retainer portion may be substantially greater than a diameter of the fluid sealing plug. For example, in the implanted configuration, the diameter of the distal retainer portion may be about 1.25 to about 3.0 times of at least one of a diameter of the opening and a diameter of the fluid sealing plug.

The fluid sealing plug may have a generally toroidal shape about a longitudinal axis of the closure device. The generally toroidal shape of the fluid sealing plug may define a central, axial channel, the channel receiving at least a portion of the central retainer portion therein. The retainer may include a retainer wire structure including a proximal retainer wire portion forming the proximal retainer portion, a distal retainer wire portion forming the distal retainer portion, and a central retainer wire portion forming the central retainer portion. In an implanted configuration, the proximal retainer wire portion may be generally in the form of a circular loop. In an implanted configuration, the distal retainer wire portion may be generally in the form of a circular loop. In an implanted configuration, at least one of the proximal retainer wire portion and the distal retainer wire portion may be generally in the form of a polygon. In an implanted configuration, the proximal retainer wire portion may be disposed generally in a plane oriented substantially perpendicular to a longitudinal axis of the closure device. In an implanted configuration, the distal retainer wire portion may be disposed generally in a plane oriented substantially perpendicular to a longitudinal axis of the closure device. The retainer wire structure may be unitary and continuous across the proximal retainer wire portion, the distal retainer wire portion, and the central retainer wire portion. The proximal retainer wire portion may include a first free end of the retainer wire structure and the distal retainer wire portion may include a second free end of the retainer wire structure. In an implanted configuration, the central retainer wire portion may extend generally longitudinally axially between the proximal retainer wire portion and the distal retainer wire portion. In an implanted configuration, the central retainer wire portion may extend transversely with respect to a longitudinal axis of the closure device between the proximal retainer wire portion and the distal retainer wire portion. In an implanted configuration, the central retainer wire portion may extend substantially obliquely with respect to a longitudinal axis of the closure device between the proximal retainer wire portion and the distal retainer wire portion.

At least one of a proximal retainer portion cover at least partially enclosing the proximal retainer wire portion and a distal retainer portion cover at least partially enclosing the distal retainer wire portion may be provided for the device. At least one of the proximal retainer portion cover and the distal retainer portion cover may include a porous polymer material. As another feature, the device may include both the proximal retainer portion cover and the distal retainer portion cover. The retainer wire structure may include a proximal connecting portion between the proximal retainer wire portion and the central retainer wire portion and the proximal connecting portion may be disposed at a formed angle relative to the proximal retainer wire portion. The retainer wire structure may include a distal connecting portion between the distal retainer wire portion and the central retainer wire portion and the distal connecting portion may be disposed at a formed angle relative to the distal retainer wire portion. The device may include a marker configured to be detectable using a medical imaging technique. The biologic tissue membrane may be a meninges and the biological fluid may be cerebrospinal fluid.

Generally, a closure device delivery system including a closure device is provided, the closure device delivery system including one or more features disclosed herein and a loading device comprising a generally longitudinal lumen containing the closure device. The loading device may include a pusher assembly configured to deploy the closure device from the loading device. The pusher assembly may include a coupler releasably connected to a free end of the proximal retainer portion.

In another general aspect, an implantable closure device for sealing an opening through a biologic tissue membrane against leakage of biological fluid is provided. The closure device includes a fluid sealing plug configured to be positioned at least partially within the opening through a biologic tissue membrane, the fluid sealing plug having a diameter. A retainer is configured to secure the fluid sealing plug at least partially within the opening. The retainer includes a proximal retainer portion having a proximal retainer portion diameter. The proximal retainer portion is configured to be disposed proximally on the biologic tissue membrane. A distal retainer portion has a distal retainer portion diameter. The distal retainer portion is configured to be disposed distally on the biologic tissue membrane. A central retainer portion is coupled to the fluid sealing plug and is configured to extend through the opening and the fluid sealing plug. The central retainer portion connects the proximal retainer portion and the distal retainer portion. The retainer is reconfigurable between an implanted configuration in which the proximal retainer portion diameter is greater than the diameter of the fluid sealing plug and the distal retainer portion diameter is greater than the diameter of the fluid sealing plug, and a delivery configuration in which distal retainer portion diameter is approximately equal to or less than the diameter of the fluid sealing plug.

The closure device may have various optional or additional features. In the delivery configuration, the proximal retainer portion diameter may be approximately equal to or less than the diameter of the fluid sealing plug. The proximal retainer portion may at least partially define a proximal retainer portion central area and the proximal retainer portion central area may be substantially open. The distal retainer portion may at least partially define a distal retainer portion central area and the distal retainer portion central area may be substantially open. The retainer may include a retainer wire structure including a proximal retainer wire portion forming the proximal retainer portion, a distal retainer wire portion forming the distal retainer portion, and a central retainer wire portion forming the central retainer portion. In an implanted configuration, at least one of the proximal retainer wire portion and the distal retainer wire portion may be generally in the form of a circular loop. In an implanted configuration, at least one of the proximal retainer wire portion and the distal retainer wire portion may be generally in the form of a polygon. The retainer wire structure may be constructed from a superelastic material such as a metal alloy. The biologic tissue membrane may be a meninges and the biological fluid may be cerebrospinal fluid.

In another aspect, a closure device delivery system is provided. The closure device delivery system including a closure device including one or more of the features disclosed herein, a generally tubular loading device containing the closure device, the closure device being in the delivery configuration when contained in the loading device, and a pusher assembly configured to deploy the closure device from the loading device. The closure device may be configured to transition from the delivery configuration to the implanted configuration as it is deployed from the loading device. The proximal retainer wire portion may be releasably coupled to the pusher assembly. A delivery sheath assembly may be provided including a lumen extending therethrough. The loading device and the delivery sheath assembly may be configured to be releasably coupled together such that the lumen of the delivery sheath assembly is generally axially aligned with a lumen of the loading device. A needle assembly may be provided and the needle assembly and the delivery sheath assembly may be configured to be releasably coupled together such that at least a portion of the needle assembly extends through the lumen of the delivery sheath assembly.

Generally, a method for closing an opening through biologic tissue is provided. A method for closing an opening through a biologic tissue membrane and sealing against leakage of biological fluid includes deploying a distal retainer portion of a closure device distal to a distal surface of a biologic tissue membrane through an opening through the biologic tissue membrane. A fluid sealing plug coupled to a central retainer portion of the closure device is deployed at least partially into the opening. A proximal retainer portion of the closure device is deployed proximal to a proximal surface of the biologic tissue membrane. The method may include various additional or optional features or methodology.

Deploying the distal retainer portion of the closure device may include transitioning the distal retainer portion from a delivery configuration to an implanted configuration. Deploying the proximal retainer portion of the closure device may include transitioning the proximal retainer portion from a delivery configuration to an implanted configuration. Deploying the distal retainer portion of the closure device may include deploying the distal retainer portion distally spaced apart from the distal surface of the biologic tissue membrane. Prior to deploying the fluid sealing plug, the closure device may be withdrawn to place the distal retainer portion in contact with the distal surface of the biologic tissue membrane. Before deploying the distal retainer portion of the closure device, a generally tubular delivery sheath assembly may be advanced through the opening, and a loading device may be positioned on the delivery sheath assembly, the loading device including a lumen containing the closure device in a delivery configuration. Deploying the distal retainer portion of the closure device, deploying the fluid sealing plug, and deploying the proximal retainer portion may include deploying the closure device from the loading device and through the delivery sheath assembly. Deploying the distal retainer portion of the closure device may include extending the distal retainer portion of the closure device from a distal tip of the delivery sheath assembly and transitioning the distal retainer portion from the delivery configuration to an implanted configuration. Deploying the proximal retainer portion of the closure device may include extending the proximal retainer portion of the closure device from a distal tip of the delivery sheath assembly and transitioning the proximal retainer portion from the delivery configuration to an implanted configuration. Deploying the closure device from the loading device may include advancing distally a pusher assembly, the pusher assembly being configured to push the closure device distally from the lumen of the loading device and through a lumen of the delivery sheath assembly. Deploying the proximal retainer portion of the closure device may include detaching the proximal retainer portion of the closure device from the pusher assembly.

As another optional aspect of the method, prior to detaching the proximal retainer portion from the pusher assembly, at least one of a position of the closure device and an efficacy of the closure device may be assessed. Deploying the fluid sealing plug may include withdrawing the delivery sheath assembly from the opening. Advancing the generally tubular delivery sheath assembly through the opening through the biologic tissue membrane may include advancing together the delivery sheath assembly and a needle assembly extending through the lumen of the delivery sheath assembly at least until a tip of the delivery sheath assembly has penetrated the biologic tissue membrane. Before positioning the loading device on the delivery sheath assembly, the needle assembly may be removed from the delivery sheath assembly. The biologic tissue membrane may be a meninges and the biological fluid may be cerebrospinal fluid.

Additional features, options or aspects of the invention will become more apparent through a review of a detailed description of various illustrative embodiments described in more detail herein, taken in conjunction with the accompanying drawings of these illustrative embodiments.

### BRIEF DESCRIPTION OF THE DRAWINGS

FIG. 1 is an isometric view of a closure device in a biologic tissue membrane and constructed according to a first illustrative embodiment.

FIG. 2 is another perspective view of the closure device of FIG. 1.

FIG. 3 is another perspective view of the closure device of FIG. 1 shown with optional proximal and distal retainer portion covers.

FIG. 4 is an exploded isometric view of the exemplary closure device of FIG. 3.

FIG. 5 is a perspective view of the exemplary closure device of FIG. 1 shown with an optional proximal retainer portion cover.

FIG. 6 is an elevation view of a retainer wire structure of the exemplary closure device of FIG. 1.

FIG. 7 is a plan view of the retainer wire structure of the exemplary closure device of FIG. 1.

FIG. 8 is a perspective view of a retainer wire structure for a closure device constructed according to another illustrative embodiment.

FIG. 9 is an elevation view of the illustrative retainer wire structure of FIG. 8.

FIG. 10 is a plan view of the illustrative retainer wire structure of FIG. 8.

FIG. 11A is a plan view of an alternative illustrative retainer wire structure.

FIG. 11B is an elevation view of an alternative illustrative retainer wire structure.

FIG. 12 is a sectional view illustrating an initial step of an illustrative method for implanting the closure device of FIG. 1 in a biologic tissue membrane.

FIG. 13 is a sectional view illustrating a further step of the illustrative method for implanting the closure device of FIG. 1.

FIG. 14 is a sectional view illustrating a further step of the illustrative method for implanting the closure device of FIG. 1.

FIG. 15 is a sectional view illustrating a further step of the illustrative method for implanting the closure device of FIG. 1.

FIG. 16 is a sectional view illustrating a further step of the illustrative method for implanting the closure device of FIG. 1.

FIG. 17 is a sectional view illustrating a further step of the illustrative method for implanting the closure device of FIG. 1.

FIG. 18 is a sectional view illustrating a further step of the illustrative method for implanting the closure device of FIG. 1.

FIG. 19 is a top perspective view of the device of FIG. 1 after implantation.

FIG. 20 is a sectional view illustrating a final step in the illustrative method for implanting the device of FIG. 1.

FIG. 21 is a sectional view of the implanted device of FIG. 1.

### DETAILED DESCRIPTION

Illustrative embodiments according to at least some aspects of the present disclosure are described and illustrated below and include devices and methods relating to medical procedures. It will be apparent to those of ordinary skill in the art that the embodiments discussed below are examples and may be reconfigured without departing from the scope and spirit of the present disclosure. It is also to be understood that variations of the exemplary embodiments contemplated by one of ordinary skill in the art shall concurrently comprise part of the instant disclosure. The illustrative embodiments as discussed below may include optional steps, methods, and features that one of ordinary skill should recognize as not being a requisite to fall within the scope of the present disclosure.

The present disclosure includes, inter alia, implantable tissue closure devices. Some illustrative embodiments according to at least some aspects of the present disclosure may be used as implantable closure devices for openings such as punctures, or holes, in biological tissue such as the meninges membranes. Some illustrative embodiments may reduce and/or prevent leakage of biological fluid, such as cerebrospinal fluid, through an opening such as a puncture, e.g., into the soft tissue space (e.g., fat, skin, and/or muscle) that is superficial to the meninges membranes and the cerebrospinal fluid system. Generally, some illustrative embodiments may include a fluid sealing plug and/or a retainer. Some illustrative embodiments may be reconfigurable between a contracted low-profile, delivery configuration for insertion into the puncture or other opening and/or an expanded, higher profile implanted configuration in which the fluid sealing plug at least partially obstructs the puncture or opening and/or the retainer secures the fluid sealing plug in position. While the present detailed description of illustrative embodiments refers to punctures which are generally made during surgical treatment, it will be appreciated that other tissue openings such as natural defects or surgical openings and tissue injuries may be sealed as well.

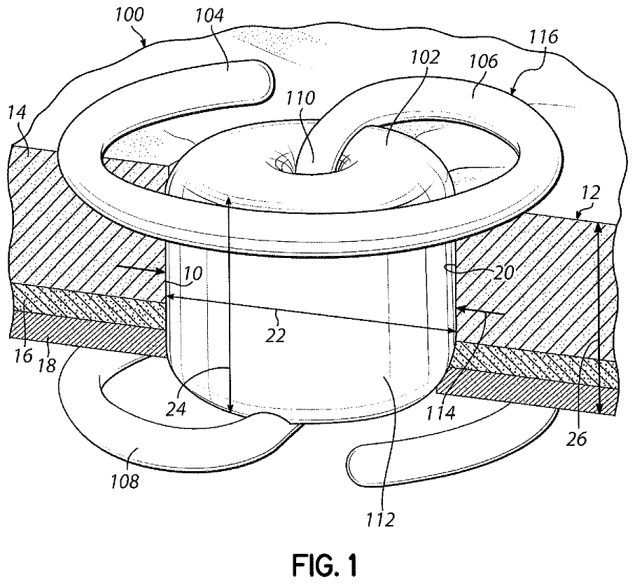

FIG. 1 is an isometric, partial sectional view of an illustrative closure device 100 implanted in a puncture 10 through a biologic tissue membrane, such as the meninges 12, according to at least some aspects of the present disclosure. Generally, the closure device 100 may be configured to seal against leakage of a biological fluid (e.g., the cerebrospinal fluid) through the sealed opening of the tissue (e.g., the puncture 10). The meninges 12 includes three layers: dura mater 14 (outer/superficial layer), arachnoid mater 16 (middle layer), and pia mater 18 (inner/deep layer). Although some illustrative embodiments are described herein in connection with the meninges and cerebrospinal fluid, it is within the scope of the present disclosure to utilize various illustrative closure devices in connection with other biologic tissue membranes and/or to seal openings against leakage of other biological fluids.

The illustrative closure device 100 includes a fluid sealing plug 102, which may be configured to be positioned at least partially within the puncture 10. The closure device 100 includes a retainer 104, which may be configured to secure the plug 102 in a desired position, such as at least partially within the puncture 10. For example, a proximal retainer portion 106 of the retainer 104 may be disposed proximally on the meninges 12 (e.g., on an outwardly, superficially facing surface of the dura mater 14) and/or a distal retainer portion 108 of the retainer 104 may be disposed distally on the meninges 12 (e.g., on an inwardly, deep facing surface of the pia mater 18). As used herein, “distal” may refer generally to the direction towards the center of a patient's body, and “proximal” may refer generally to the direction away from the center of the patient's body. Depending on the circumstances, “proximal” also refers to a position closer to the user of the device, while “distal” refers to a position farther from the user of the device. A central retainer portion 110 of the retainer 104 may extend through the puncture 10 and/or may connect the proximal retainer portion 106 and the distal retainer portion 108. The plug 102 may be directly or indirectly coupled to the central retainer portion 110, such as between the proximal retainer portion 106 and the distal retainer portion 108.

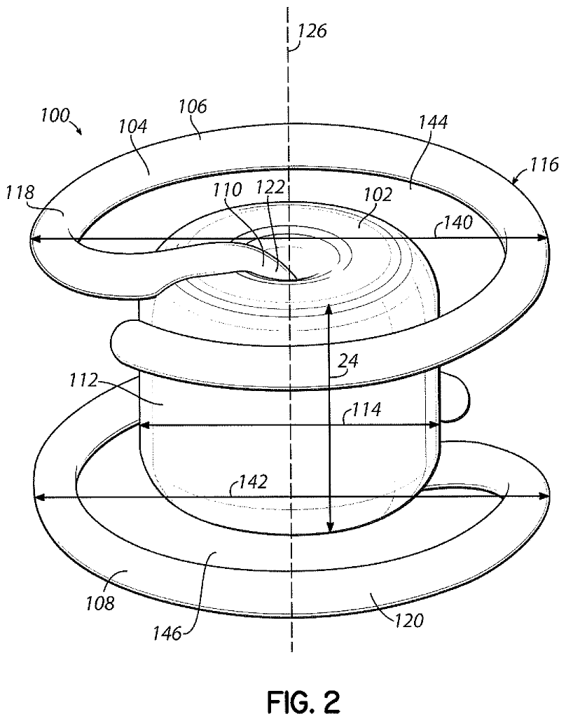

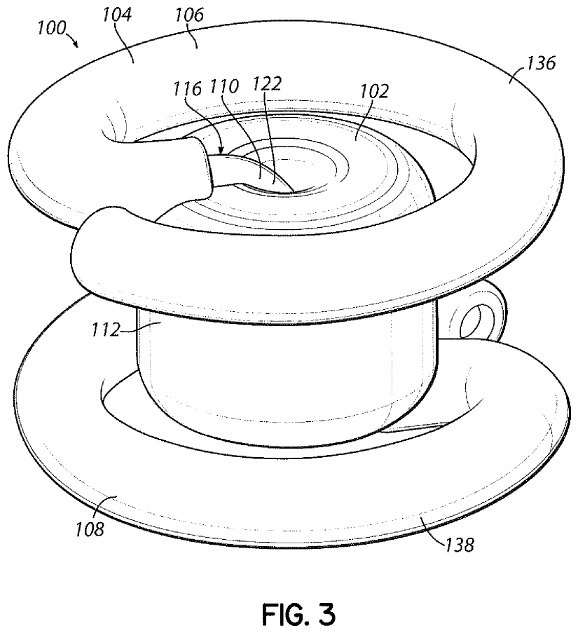

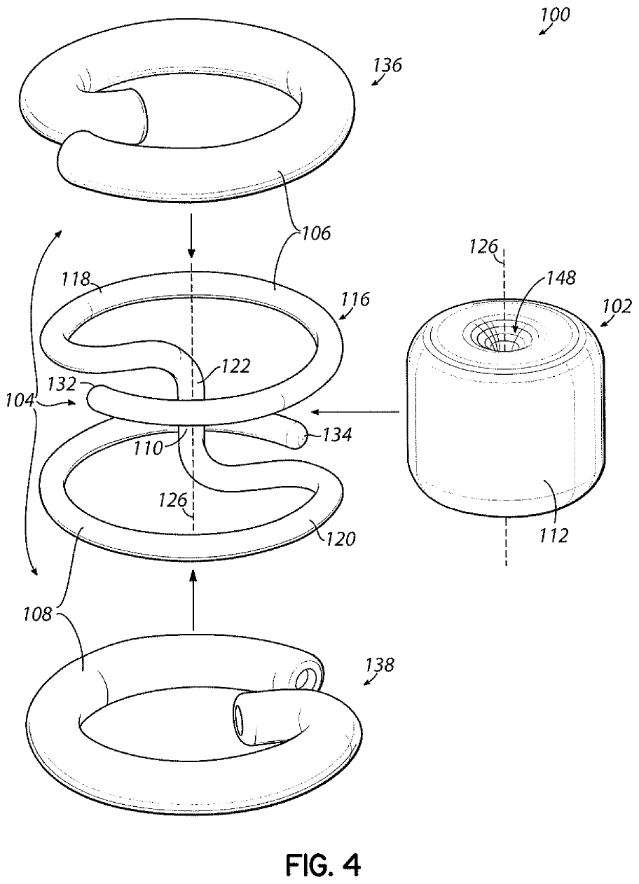

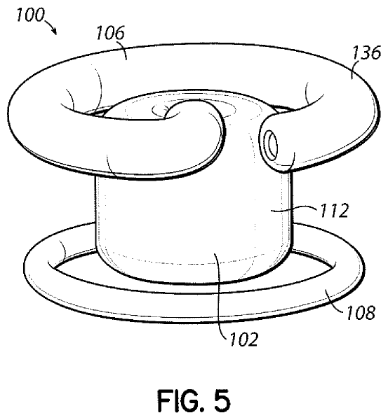

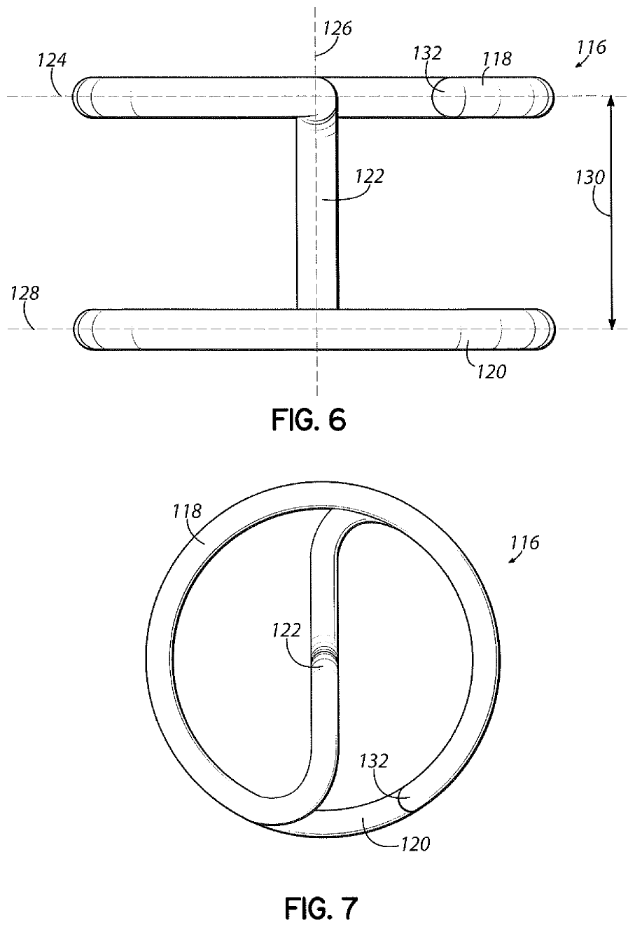

FIG. 2 is an isometric view of the exemplary closure device 100, FIG. 3 is an isometric view of the exemplary closure device 100 shown with an optional proximal retainer portion cover 136 and an optional distal retainer portion cover 138; FIG. 4 is an exploded isometric view of the exemplary closure device 100; FIG. 5 is an isometric view of the exemplary closure device 100 shown with the optional proximal retainer portion cover 136; FIG. 6 is an elevation view of a retainer wire structure 116 of the exemplary closure device 100; and FIG. 7 is a plan view of the retainer wire structure 116 of the exemplary closure device 100, all according to at least some aspects of the present disclosure.

Referring to FIG. 1, the fluid sealing plug 102 may be configured to at least partially obstruct or occlude the puncture 10 through the meninges 12. The puncture 10 may be at least partially, such as substantially or fully, sealed by the plug 102 in at least one of the dura mater 14, arachnoid mater 16, and/or pia mater 18. A generally circumferential, radial outer surface 112 of the plug 102, having a diameter 114, may engage a generally circumferential, radial inner surface 20 of the puncture 10, having a diameter 22, to provide an at least partially sealed interface between the outer surface 112 of the plug 102 and the inner surface 20 of the puncture 10. As used herein, “diameter” may refer to a major dimension of a shape generally corresponding to the diameter dimension of a circle and is not limited to circular shapes. Also, “diameter” may refer to an exterior dimension, such as the outer diameter of a generally cylindrical object, or to an interior dimension, such as the inner diameter of a tube.

Generally, the diameter 114 of the fluid sealing plug 102 may be selected to generally correspond to diameter 22 of the puncture 10. For example, the diameter 114 of the plug 102 may approximately match the diameter 22 of the puncture 10. In some alternative embodiments, the uncompressed diameter 114 of the plug 102 may be greater than the diameter 22 of the puncture 10, which may facilitate secure engagement of the plug 102 within the puncture 10. In some alternative embodiments, the diameter 114 of the plug 102 may be less than the diameter 22 of the puncture 10. The plug 102 may have an axial length 24, which may generally correspond to the thickness 26 of the tissue (e.g., meninges 12) containing the puncture 10, although some embodiments may include plugs that are substantially longer or shorter than the thickness 26 of the tissue containing the puncture 10.

In some exemplary embodiments, the fluid sealing plug 102 may be constructed of a porous polymer material, such as expanded polytetrafluoroethylene (ePTFE) or other polymer matrix formed by a process such as knitting or electrospinning. In some exemplary embodiments, the plug 102 may be at least partially compressible. In some exemplary embodiments, the plug 102 may be constructed from a material that promotes healing of the tissue around the plug 102 and/or tissue ingrowth into the plug 102, which may reinforce or increase sealing of the puncture 10.

Referring to FIGS. 2, 4, 6, and 7, the illustrative retainer 104 includes a retainer wire structure 116. In some exemplary embodiments, the retainer wire structure 116 may be pre-formed into the shape as shown. The retainer wire structure 116 includes a proximal retainer wire portion 118 (forming the proximal retainer portion 106), a distal retainer wire portion 120 (forming the distal retainer portion 108), and a central retainer wire portion 122 (forming the central retainer portion 110).

In this illustrative embodiment, in an implanted configuration, the proximal retainer wire portion 118 may be generally in the form of a circular loop. The proximal retainer wire portion 118 may be disposed generally in a plane 124, which may be oriented substantially perpendicular to a longitudinal axis 126 of the closure device 100. The distal retainer wire portion 120 may be generally in the form of a circular loop. The distal wire retainer portion 122 may be disposed generally in a plane 128, which may be oriented substantially perpendicular to the longitudinal axis 126 of the closure device 100. Accordingly, the proximal retainer wire portion 118 and the distal retainer wire portion 120 may be disposed in generally parallel planes 124, 126 spaced apart by a distance 130, which may generally correspond to the thickness of the biologic tissue membrane (e.g., meninges 12) containing the puncture 10. In an illustrative embodiment, the distance 130 may be about 0.010 inches. In this illustrative embodiment, the central retainer wire portion 122 extends generally longitudinally axially between the proximal retainer wire portion 118 and the distal retainer wire portion 120.

The proximal retainer wire portion 118 may include a first free end 132 of the retainer wire structure 116 and/or the distal retainer wire portion 120 may include a second free end 134 of the retainer wire structure 116. The free ends 132, 134 may be constructed with generally smooth (e.g., radiused or edge break) to reduce the risk of tissue trauma, such as during deployment and fixation of the closure device 100.

Referring to FIGS. 4, 6, and 7, in some illustrative embodiments, the retainer wire structure 116 may be substantially unitary and/or continuous across the proximal retainer wire portion 118, the distal retainer wire portion 120, and/or the central retainer wire portion 122. For example, the retainer wire structure 116 may be formed from a single segment of metal wire, such as a superelastic metal (e.g., a nickel titanium alloy). In some exemplary embodiments, the diameter of the wire forming the retainer wire structure 116 may be about 0.001 inches to about 0.010 inches. The wire may have a surface finish ranging from a smooth electropolished finish to a generally rougher, sintered finish. In some exemplary embodiments, one or more of the proximal retainer wire portion 118, the distal retainer wire portion 120, and/or the central retainer wire portion 122 may have a different thickness or cross-section than one or more of the others, which may facilitate different desired closure characteristics on the adjacent tissues.

Referring to FIGS. 3 and 4, in some illustrative embodiments, the proximal retainer portion 106 may include a proximal retainer portion cover 136 and/or the distal retainer portion 108 may include a distal retainer portion cover 138. For example, the proximal retainer portion cover 136 may be generally in the form of a tube that at least partially encloses the proximal retainer wire portion 118 and/or the distal retainer portion cover 138 may be generally in the form of a tube that at least partially encloses distal retainer wire portion 120. The proximal retainer portion cover 136 and/or the distal retainer portion cover 138 may be constructed of a porous polymer material, for example. FIG. 5 depicts an illustrative embodiment in which the proximal retainer portion 106 includes a proximal retainer portion cover 136 and the distal retainer portion 108 does not include a distal retainer portion cover 138 (FIGS. 3 and 4). The proximal retainer portion cover 136 and/or the distal retainer portion cover 138 may aid in securing the closure device 100 on the meninges and/or may encourage tissue growth around the closure device 100.

Referring to FIG. 2, the proximal retainer portion 106 may have a proximal retainer portion diameter 140 and/or the distal retainer portion 108 may have a distal retainer portion diameter 142. In an implanted configuration (e.g., as illustrated in FIGS. 1-7), the proximal retainer portion diameter 140 and/or the distal retainer portion diameter 142 may be substantially greater than the diameter 114 of the fluid sealing plug 102. For example, the diameters 140, 142 may be about 1.25 to about 3.0 times the diameter 114 of the plug 102. In a delivery configuration (e.g., as described below), the proximal retainer portion diameter 140 and/or the distal retainer portion diameter 142 may be less than or equal to the diameter 114 of the plug 102. In various exemplary embodiments, the proximal retainer wire portion 118 and the distal wire retainer portion 120 may have diameters 140, 142 of about 1.25 to about 3 times the diameter 22 of the puncture 10 through the meninges 12. In embodiments including retainer portion covers 136, 138 (e.g., FIG. 3), the diameters 140, 142 of the respective portions 106, 108 may be defined by the outermost major dimensions including the retainer portion covers 136, 138. In some illustrative embodiments, the diameters 140, 142 may be about 0.060 inches to about 0.160 inches. In some embodiments, the diameters 140, 142 may be approximately the same. In other embodiments, the diameters 140, 142 may be substantially different, such as to provide different characteristics with respect to the adjacent tissue or biologic tissue membranes.

Referring to FIG. 2, the proximal retainer portion 106 may at least partially define a proximal retainer portion central area 144, such as generally radially within the outer periphery of the proximal retainer portion 106. In this illustrative embodiment, the proximal retainer portion central area 144 is substantially open (e.g., substantially unobstructed). Similarly, the distal retainer portion 108 may at least partially define a distal retainer portion central area 146, such as generally radially within the outer periphery of the distal retainer portion 106. In this illustrative embodiment, the distal retainer portion central area 146 is substantially open (e.g., substantially unobstructed). Accordingly, in the illustrative embodiment shown in FIGS. 1-7, the puncture 10 is substantially occluded by the fluid sealing plug 102 rather than any components extending across the central areas 144, 146.

In alternative embodiments (not shown) including a closed proximal retainer portion central area 144 and/or a closed distal retainer portion central area 146, the puncture 10 may be at least partially occluded by a structure substantially broader than the puncture 10 extending across a central area 144, 146 and covering the puncture 10 and a substantial portion of the surface of the biologic tissue membrane (e.g., meninges 12) surrounding the puncture 10. For example, some such alternative embodiments may include a thin, generally planar portion (e.g., a disc) of biologically compatible material having a diameter substantially greater than the diameter 22 of the puncture 10. The disc may be positioned against one side of the meninges 12 such that the disc covers the puncture 10 and a substantial portion of the surface of the meninges 12, thereby preventing flow of fluid through the puncture 12. Some embodiments may include such a disc on both sides of the meninges 12.

Referring to FIG. 4, the fluid sealing plug 102 may have a generally toroidal shape about the longitudinal axis 126 of the closure device 100, including a central, axial channel 148. In some exemplary embodiments, the channel 148 may receive at least a portion of the central retainer portion 110 of the retainer 104, such as the central retainer wire portion 122, when the closure device is assembled. In some exemplary embodiments, regardless of whether the plug 102 is constructed from a compressible material, the plug 102 may be substantially solid. As used in this context, “solid” refers to a three-dimensional shape that is substantially filled within its exterior shape (e.g., lacks substantial internal voids). In some exemplary embodiments, the plug 102 may be at least partially hollow. As used in this context, “hollow” refers to a three-dimensional shape that includes at least one internal void. In some exemplary embodiments, the plug 102 may be constructed of two or more materials (or two or more forms of the same material) having different characteristics. For example, the plug 102 may be non-uniform radially and/or axially. Generally, regardless of its particular form, a fluid sealing plug 102 according to at least some aspects of the present disclosure may reduce and/or prevent fluid flow from one side of the biologic tissue membrane (e.g., meninges 12) to the other side of the biologic tissue membrane through the puncture 10 by sealingly engaging the puncture 10 between the sides of the biologic tissue membrane.

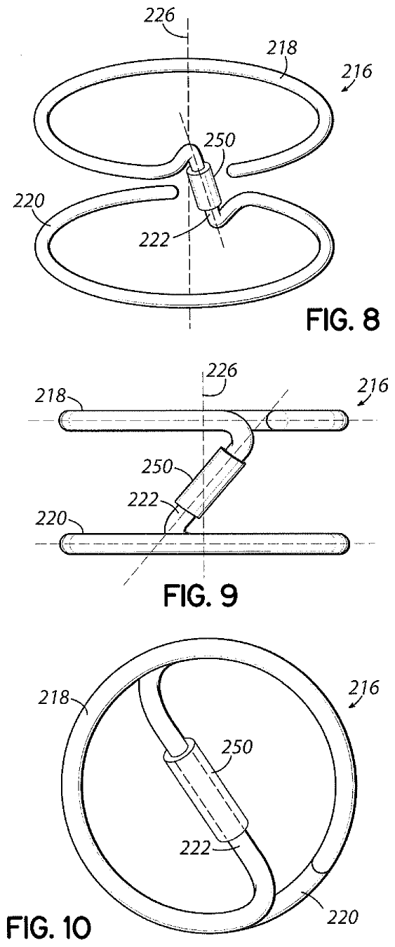

FIG. 8 is an isometric view of an alternative illustrative retainer wire structure 216; FIG. 9 is an elevation view of the alternative illustrative retainer wire structure 216 of FIG. 8; and FIG. 10 is a plan view of the alternative illustrative retainer wire structure 216 of FIG. 8, all according to at least some aspects of the present disclosure. The retainer wire structure 216 shown and described with respect to FIGS. 8-10 is generally similar to the retainer wire structure 116 shown and described with respect to FIGS. 1-7. Generally, the retainer wire structure 216 may be substituted for the retainer wire structure 116 in various embodiments according to at least some aspects of the present disclosure, such as the closure device 100 described above. Like reference numerals refer to like structure shown and described above. Unless specifically indicated, the description of the structure and function or methodology of corresponding components with respect to the retainer wire structure 116 generally applies to the retainer wire structure 216. Therefore, repeated explanation of previously described structure and function or methodology is not necessary.

In this illustrative embodiment, the central retainer wire portion 222 extends generally substantially obliquely with respect to a longitudinal axis 226 of the closure device between the proximal retainer wire portion 218 and the distal retainer wire portion 220. As used herein, “obliquely” may refer to relative angular orientations that are neither perpendicular nor parallel so that the respective components are generally slanted with respect to each other. The central retainer wire portion 222 may extend transversely with respect to the longitudinal axis 226 of the closure device between the proximal retainer wire portion 218 and the distal retainer wire portion 220. As used herein, “transversely” may refer to relative angular orientations that are non-parallel (e.g., perpendicular or oblique).

FIGS. 8-10 also show an optional marker 250, which may be used in connection with various embodiments according to at least some aspects of the present disclosure. The marker 250 may be constructed of a material that is substantially detectable, and thus visible to a user, utilizing a medical imaging technique. For example, the marker 250 may be constructed of a radiopaque material for use in connection with fluoroscopic imaging techniques and/or an echogenic material for use in connection with ultrasound imaging techniques. In some example embodiments, the marker 250 may be configured so that both its position and orientation may be determined via the medical imaging technique, such as by including non-symmetric and/or non-uniform geometric features. Accordingly, the position and/or orientation of the closure device 100 may be ascertained using the imaging technique.

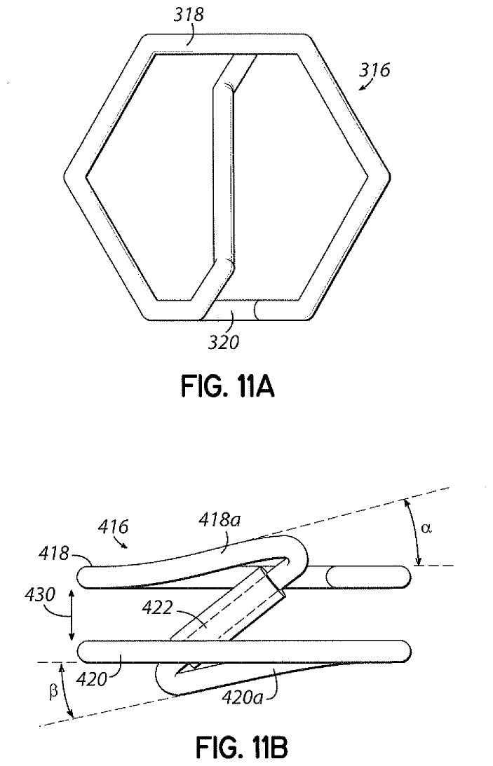

FIG. 11A is a plan view of an alternative illustrative retainer wire structure 316, according to at least some aspects of the present disclosure. The retainer wire structure 316 shown and described with respect to FIG. 11A is generally similar to the retainer wire structures 116, 216 shown and described above. Generally, the retainer wire structure 316 may be substituted for the retainer wire structures 116, 216 in various embodiments according to at least some aspects of the present disclosure, such as the closure device 100 described above. Like reference numerals refer to like structure shown and described above. Unless specifically indicated, the description of the structure and function or methodology of corresponding components with respect to the retainer wire structures 116, 216 generally applies to the retainer wire structure 316. Therefore, repeated explanation of previously described structure and function or methodology is not necessary. The retainer wire structure 316 of FIG. 11A includes a generally polygonal (e.g., hexagonal) proximal retainer wire portion 118 (forming the proximal retainer portion 106) and a generally polygonal (e.g., hexagonal) distal retainer wire portion 120 (forming the distal retainer portion 108).

FIG. 11B is an elevation view of an alternative illustrative retainer wire structure 416, according to at least some aspects of the present disclosure. The retainer wire structure 416 shown and described with respect to FIG. 11B is generally similar to the retainer wire structures 116, 216, 316 shown and described above. Generally, the retainer wire structure 416 may be substituted for the retainer wire structures 116, 216, 316 in various embodiments according to at least some aspects of the present disclosure, such as the closure device 100 described above. Like reference numerals refer to like structure shown and described above. Unless specifically indicated, the description of the structure and function or methodology of corresponding components with respect to the retainer wire structures 116, 216, 316 generally applies to the retainer wire structure 416. Therefore, repeated explanation of previously described structure and function or methodology is not necessary.

The retainer wire structure 416 of FIG. 11B includes a proximal retainer wire portion 418 (e.g., forming the proximal retainer portion 106) and a distal retainer wire portion 420 (e.g., forming the distal retainer portion 108) connected by a central retainer wire portion 422. The proximal retainer wire portion 418 and the distal retainer wire portion 420 may be disposed in generally parallel planes spaced apart by a distance 430, which may be less than the thickness of the biologic tissue membrane (e.g., meninges 12) containing the puncture 10. The illustrative retainer wire structure 416 includes a proximal connecting portion 418a between the proximal retainer wire portion 418 and the central retainer wire portion 422. The proximal connecting portion 418a is disposed at a formed angle α relative to the proximal retainer wire portion 418. Similarly, the retainer wire structure 416 includes a distal connecting portion 420a between the distal retainer wire portion 420 and the central retainer wire portion 422. The distal connecting portion 420a is disposed at a formed angle β relative to the distal retainer wire portion 420. Other exemplary embodiments may include only one connecting portion 418a, 420a having a formed angle α, β. In some exemplary embodiments, the formed angles α, β may be about zero to about 30 degrees.

When a closure device 100 including the retainer wire structure 416 is implanted in a tissue membrane that is thicker than the distance 430 between the proximal wire portion 418 and the distal retainer wire portion 420, the connecting portions 418a, 420a may elastically deform so that the tissue membrane is compressed between the proximal wire portion 418 and the distal retainer wire portion 420.

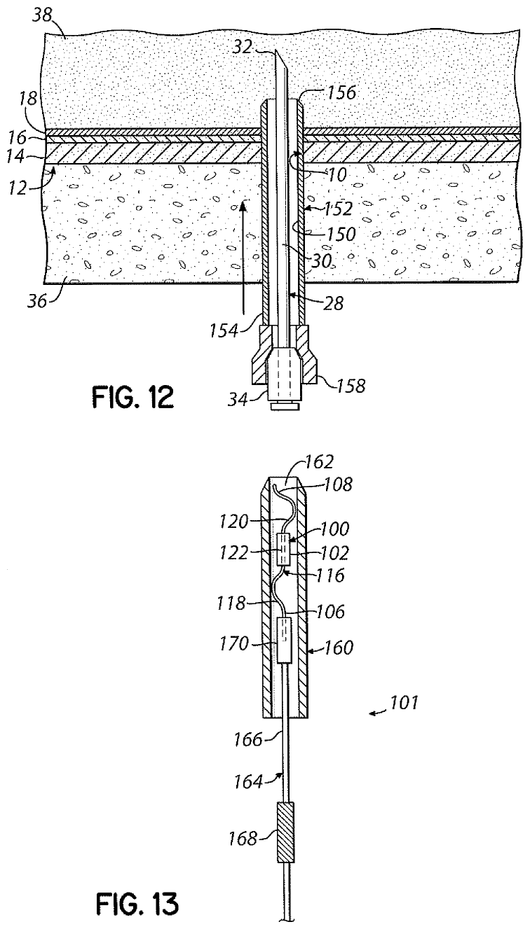

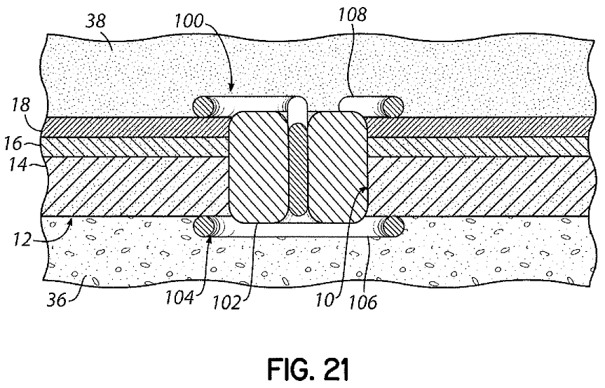

Exemplary methods of using closure devices according to the present disclosure are described below with reference to FIGS. 12-21, which may include optional and/or alternative structures and/or operations. FIGS. 12-18 and 20 are cross-section views showing illustrative operations in connection with using the closure device 100; FIG. 19 is a detailed distal isometric view of the closure device 100 positioned in the meninges 12; and FIG. 21 is a cross-section view of the closure device 100 implanted in the meninges 12, all according to at least some aspects of the present disclosure. Although FIGS. 12-21 and the corresponding description focus on the use of the closure device 100 to seal the puncture 10 through the meninges 12 to prevent and/or reduce leakage of cerebrospinal fluid, it will be appreciated that generally similar operations may be utilized when alternative embodiment closure devices are used to seal openings though other biologic tissues to prevent and/or reduce leakage of other biological fluids (e.g., blood).

Referring to FIG. 12, a needle assembly 28 is positioned in a lumen 150 of a delivery sheath assembly 152, which will be used in connection with an exemplary closure device 100 (FIG. 13). The illustrative delivery sheath assembly 152 includes a generally tubular sheath 154, which at least partially defines the lumen 150. The sheath 154 has a sheath tip 156 at a distal end of the sheath 154 and a sheath hub 158 at a proximal end of the sheath 154. In some illustrative embodiments, the sheath tip 156 may be generally rounded and/or tapered, which may facilitate insertion through tissues and/or biologic tissue membranes.

The illustrative needle assembly 28 includes an elongated needle 30 having a needle tip 32 at a distal end of the needle 30 and a needle hub 34 at a proximal end of the needle 30. The sheath 154 and the needle 30 may be sized so that a radial gap between the radial outer surface of the needle 30 and the radial inner surface of the lumen 150 of the sheath 154 is relatively small, such as about 0.001 inches to about 0.005 inches in some exemplary embodiments. Such a radially tight fit may facilitate minimal entrapment of the meninges 12 during advancement while also allowing relatively easy removal of the needle 28 from the delivery sheath assembly 152 when desired.

In use, the needle assembly 28 and the delivery sheath assembly 152 are advanced together through the soft tissue 36 superficial to the meninges 12 and through the layers 14, 16, 18 of the meninges 12 until the needle 30 and the sheath 154 extend into the cerebrospinal fluid space 38, forming the puncture 10. For example, the needle assembly 28 and the delivery sheath assembly 152 may be advanced until the needle tip 32 and/or the sheath tip 156 are within the cerebrospinal fluid space 38.

In some circumstances, it may be desirable to position the sheath assembly 152 so that the sheath tip 156 extends only a short distance into the cerebrospinal fluid space 38 (e.g., only a short distance distally beyond the deep, distal surface of the pia mater 18 of the meninges 12). In some example embodiments, this may be facilitated by constructing the needle assembly 28 and the delivery sheath assembly 152 so that the relative lengths of the needle 30 and sheath 154 and/or the interface between the needle hub 34 and the sheath hub 158 are configured to position the needle tip 32 relative to the sheath tip 156 as desired. For example, the sheath tip 156 may be positioned just proximal to opening of the lumen of the needle 30.

In some exemplary procedures, the user may access the cerebrospinal fluid space 38 via an internal lumen of the needle 30. Alternatively, the user may access the cerebrospinal fluid space 38 via the lumen 150 of the sheath 154 after removing the needle assembly 28 from the delivery sheath assembly 152. For example, the user may remove a small amount of cerebrospinal fluid for laboratory analysis and/or the user may inject a pharmaceutical into the cerebrospinal fluid space 38.

FIG. 13 shows an exemplary closure device 100 in a delivery configuration provided in a loading device 160. Generally, the closure device 100 and the loading device 160 comprise a closure device delivery system 101, which may also include the delivery sheath assembly 152 and/or the needle assembly 28. In various embodiments, exemplary closure devices 100 may be supplied to users in loading devices 160, or users may insert closure devices 100 into separately supplied loading devices 160 before use. The illustrative loading device 160 is generally tubular and includes a generally longitudinal lumen 162 extending therethrough. The lumen 162 of the loading device 160 may have an inner diameter that is approximately the same as, or slightly smaller than, the inner diameter of the lumen 150 of the sheath 154 (FIG. 12). The inner diameter of the lumen 162 of the loading device 160 may be substantially smaller than the proximal retainer portion diameter 140 and/or distal retainer portion diameter 142 of the retainer 104 (FIG. 2) when the closure device 100 is in the implanted configuration.

In the delivery configuration shown in FIG. 13, the retainer wire structure 116 (including the proximal retainer wire portion 118, the distal retainer wire portion 120, and the central retainer wire portion 122) may be arranged generally linearly within the lumen 162 of the loading device 160. The lumen 162 of the loading device 160 may constrain the closure device 100 so that, in this delivery configuration, the diameter of the proximal retainer portion 106 and/or the diameter of the distal retainer portion 108 may be approximately equal to or less than the diameter 114 of the plug 102 (FIG. 2).

As mentioned above, some exemplary closure devices 100 may include a retainer wire structure 116 constructed of a superelastic metal (e.g., a nickel titanium alloy). Some such metals may have the material characteristics necessary to allow elastic deformation of the retainer wire structure 116 from the implanted configuration (FIG. 2) to the delivery configuration (FIG. 13) and back to the implanted configuration, such as described below. Retainers constructed from alternative materials providing similar capabilities are within the scope of the present disclosure.

In some exemplary embodiments, the uncompressed diameter 114 of the fluid sealing plug 102 (FIG. 2) may be greater than the inner diameter of the lumen 162 of the loading device 160 so that the plug 102 is at least partially compressed when it is in the delivery configuration in the loading device 160. Alternatively, the uncompressed diameter 114 of the plug 102 (FIG. 2) may be approximately equal to or less than the inner diameter of the lumen 162 of the loading device 160.

The loading device 160 may include a pusher assembly 164, which may be used to deploy the closure device 100 from the loading device 160 as described below. The illustrative pusher assembly 164 includes a push rod 166, which may be constructed from a metallic wire or a rigid polymer, for example. The pusher assembly 164 also includes a stop 168 and a coupler 170 disposed on the push rod 166. The stop 168 may be removable and/or slidable with respect to the push rod 166, or it may be permanently affixed to the push rod 166. The coupler 170 releasably connects the push rod 166 to the closure device 100. Generally, the pusher assembly 164 may be used to effect distal movement of the closure device 100 relative to the loading device 160. In this illustrative embodiment, the pusher assembly 164 is configured to facilitate advancement of the closure device 100 with approximately a 1:1 ratio of movement between the user's hand on the push rod 166 and the closure device 100. Other ratios of movement may be used instead by incorporating an appropriately designed movement or drive mechanism.

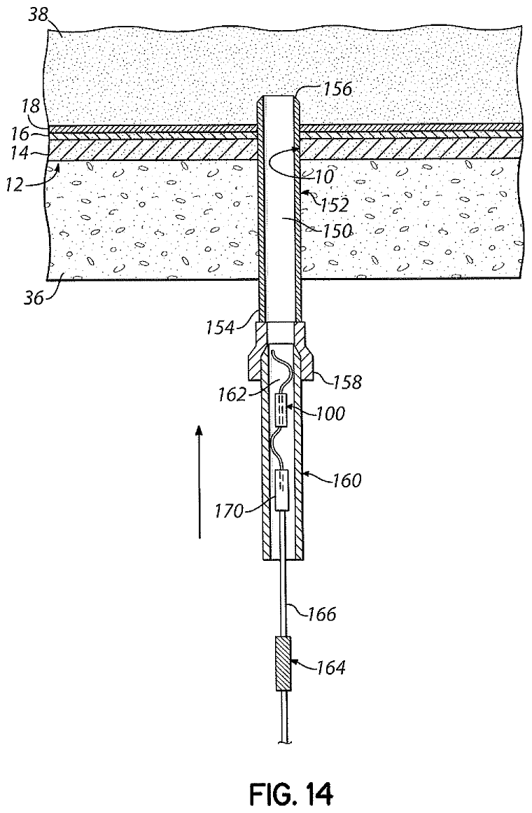

Once the need for access to the cerebrospinal fluid space 38 via the puncture 10 is finished, implantation of the closure device 100 may begin. Referring to FIG. 14, the needle assembly 28 (FIG. 12) has been removed from the delivery sheath assembly 152, leaving the sheath 154 extending through the soft tissue 36 and the meninges 12 and into the cerebrospinal fluid space 38 through the puncture 10. Generally, the sheath 154 may be maintained in this position until specifically withdrawn as described below.

The loading device 160, containing the closure device 100, is positioned on the delivery sheath assembly 152 so that the lumen 162 of the loading device 160 is generally axially aligned with the lumen 150 of the sheath 154. For example, the distal end of the loading device 160 may be inserted into the cavity of the sheath hub 158, and/or corresponding engaging features may be used to releasably couple the delivery sheath assembly 152 and the loading device 160.

Deployment of the closure device 100 is begun by the user pushing distally on pusher assembly 164 (e.g., the push rod 166), which pushes the closure device 100 distally with the lumen 162 of the loading device 160 via the coupler 170. Further advancement of the pusher assembly 164 advances the closure device 100 distally into the lumen 150 of the delivery sheath assembly 152.

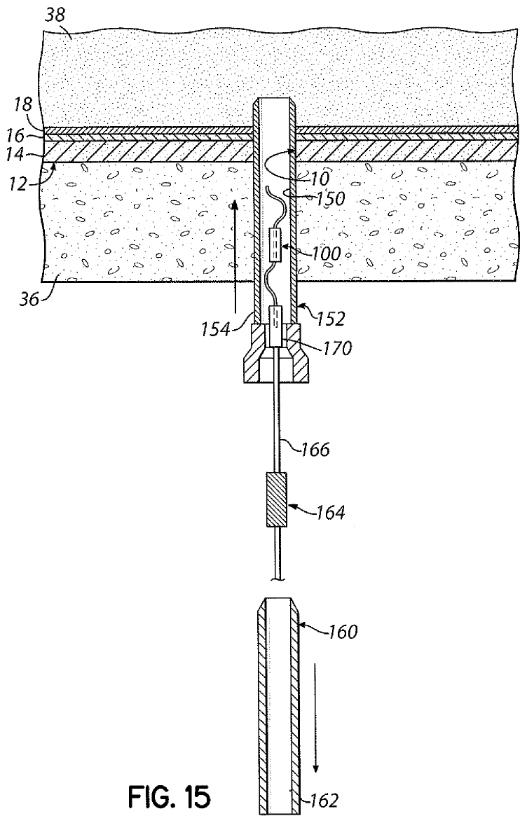

Referring to FIG. 15, the pusher assembly 164 is advanced until the closure device 100 and/or the coupler 170 are substantially out of the lumen 162 of the loading device 160 and/or are substantially within the lumen 150 of the delivery sheath assembly 152. Then, the loading device 160 is withdrawn distally and set aside, leaving the closure device 100 and pusher assembly 164 in place with respect to the delivery sheath assembly 152. The sheath 154 continues to extend through the soft tissue 36 and the meninges 12 and into the cerebrospinal fluid space 38 through the puncture 10.

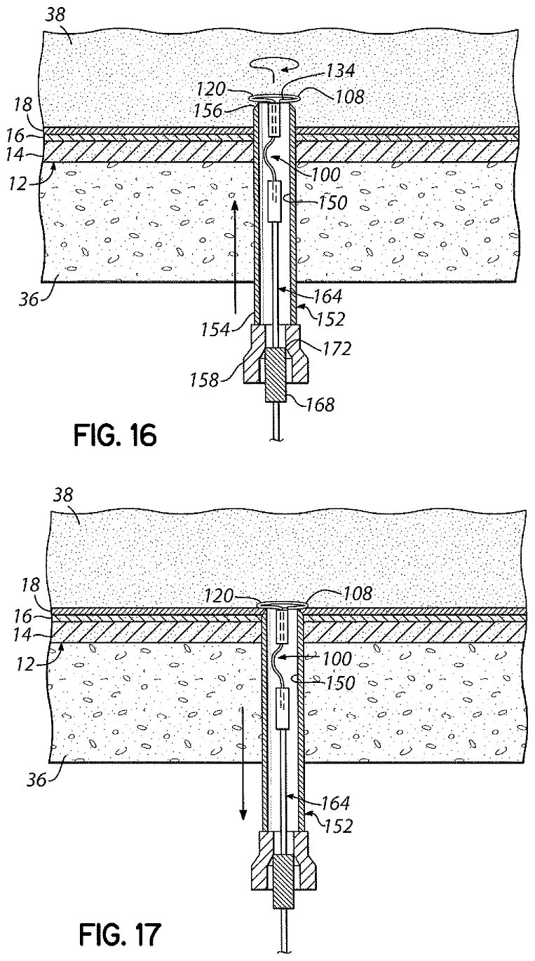

Referring to FIG. 16, the pusher assembly 164 is further advanced distally to extend the distal retainer portion 108 (e.g., the distal retainer wire portion 120) of the closure device 100 distally from the distal tip 156 of the sheath 154. For example, the second free end 134 of the distal retainer wire portion 120 may first emerge from the sheath 154 and into the cerebrospinal fluid space 38. Due to the characteristics of the material from which the retainer 104 (FIG. 2) is constructed, the distal wire retainer portion 120 returns to its implanted shape (e.g., generally circular) as it exits from and/or becomes unconstrained by the sheath 154.

Further distal advancement of the pusher assembly 164 and/or the closure device 100 relative to the delivery sheath assembly 152 is prevented by engagement of the stop 168 of the pusher assembly 164 with a corresponding stop feature of the delivery sheath assembly 152, such as a shoulder 172 disposed within the cavity of the sheath hub 158.

In this illustrative embodiment, the distal tip 156 of the sheath 154 extends distally beyond the deep, distal surface of the pia mater 18 in the cerebrospinal fluid space 38. Accordingly, the distal retainer wire portion 120 of the closure device 100 is deployed slightly distally spaced apart from the surface of the pia mater 18.

Referring to FIG. 17, the distal retainer portion 108 (e.g., the distal retainer wire portion 120) of the closure device 100 is placed into contact with the deep, distal surface of the pia mater 18, such as by withdrawing together the delivery sheath assembly 152 and the pusher assembly 164 while substantially maintaining their positions relative to each other. Engagement of the distal retainer portion 108 against the deep, distal surface of the pia mater 18 may be felt by the user and/or may be observed using an imaging device.

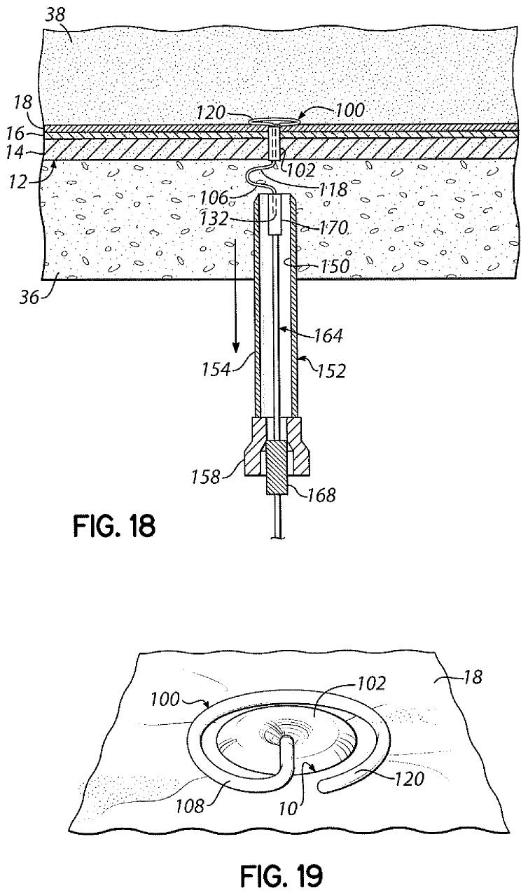

Referring to FIG. 18, the pusher assembly 164 is adjusted to allow further distal movement of the closure device 100 relative to the delivery sheath assembly 152. For example, the stop 168 may be removed from the push rod 166, the stop 168 may be moved proximally (e.g., by unlocking and sliding) on the push rod 166, or the pusher assembly 164 and/or the shoulder 172 may be repositioned to allow the stop 168 to pass by the shoulder 172. Then, the fluid sealing plug 102 and the proximal retainer portion 106 (e.g., the proximal retainer wire portion 118) may be deployed distally out of the sheath 154 by moving the pusher assembly 164 distally relative to the sheath 154. For example, the delivery sheath assembly 152 may be withdrawn proximally from the meninges 12 while maintaining the pusher assembly 164 substantially in place relative to the meninges 12. Due to the characteristics of the material from which the retainer 104 (FIG. 2) is constructed, the proximal wire retainer portion 118 returns to its implanted shape (e.g., generally circular) as it exits from and/or becomes radially unconstrained by the sheath 154. As the proximal wire retainer portion 118 emerges from the sheath 154 and returns to its implanted shape, the proximal retainer wire portion 118 contacts the superficial, proximal surface of the dura mater 14.

Referring to FIG. 19, the distal retainer portion 108 (e.g., the distal retainer wire portion 120) may be disposed substantially against the deep, distal surface of the pia mater 18. The fluid sealing plug 102 may at least partially (e.g., substantially or fully) occlude the puncture 10. Referring to FIGS. 18 and 19, the meninges 12 may be compressed between the deployed portion of the proximal wire retainer portion 118 and the distal wire retainer portion 120.

At this point, the closure device 100 is substantially deployed (e.g., implanted in the meninges 12 to close the puncture 10), except that the free end 132 of the proximal retainer portion 106 (e.g., the proximal wire retainer portion 118) remains connected to the coupler 170 of the pusher assembly 164. Accordingly, if necessary, the closure device 100 may still be removed from the meninges and/or retracted into the delivery sheath assembly 152 by withdrawing proximally the pusher assembly 164 and then withdrawing the delivery sheath assembly 152, pusher assembly 164, and closure device 100 from the patient.

In some procedures, the position of the closure device 100 may be verified and/or the closure device 100 may be monitored to determine its efficacy of closing the puncture 10. If the closure device 100 is not positioned as desired and/or does not satisfactorily seal the puncture 10, the closure device 100 may be removed.

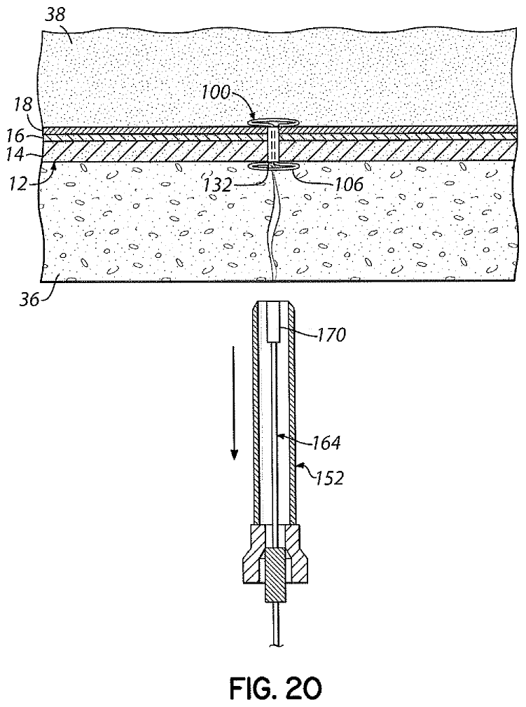

Referring to FIG. 20, once the user is satisfied with the implantation of the closure device 100, the free end 132 of the proximal retainer portion may be detached from the coupler 170 of the pusher assembly 164. For example, if the free end 132 is retained by the coupler 170 through a frictional fit, the pusher assembly 164 may be withdrawn proximally to detach the free end 132 from the coupler 170. In alternative embodiments employing an actuatable release mechanism in the coupler 170, the mechanism may be actuated to release the free end 132 from the coupler 170. Once the free end 132 is detached from the coupler 170, the proximal retainer portion 106 may fully return to its implanted configuration. The delivery sheath assembly 152 and the pusher assembly 164 are withdrawn from the patient. The wound remaining in the soft tissue 36 may seal spontaneously, such as due to the elastic properties of the tissue 36. In some procedures, the wound may be closed (e.g., sutured) and/or bandaged.

Referring to FIG. 21, when implanted, the proximal retainer portion 106 may be disposed substantially against the superficial, proximal surface of the dura mater 14. The distal retainer portion 108 may be disposed substantially against the deep, distal surface of the pia mater 18. The meninges 12 may be secured substantially between the proximal retainer portion 106 and the distal retainer portion 108 of the retainer 104. Accordingly, the retainer 104 may substantially secure the fluid sealing plug 102 at least partially within the puncture 10 through the meninges 12. The plug 102 may at least partially occlude the puncture 10 through the meninges. For example, the plug may substantially or fully seal the puncture 10 through the meninges 12, such as to prevent substantial leakage of cerebrospinal fluid from the cerebrospinal fluid space 38 to the soft tissue 36 through the puncture 10. In various embodiments, the wound or puncture 10 may seal even more fully against fluid leakage as the wound heals with the closure device 100 in place, such as described herein.

Various steps of the delivery and/or deployment process of the closure device 100 described above may be conducted using clinically acceptable visualization techniques (e.g. fluoroscopy, endoscopy, a computed tomography scan, magnetic resonance imaging, ultrasound, etc.) as desired by the user. Generally similar methods and/or structures may be used to deliver and/or deploy alternative embodiment closure devices according to at least some aspects of the present disclosure.

While the present invention has been illustrated by the description of specific embodiments thereof, and while the embodiments have been described in considerable detail, it is not intended to restrict or in any way limit the scope of the appended claims to such detail. The various features discussed herein may be used alone or in any combination within and between the various embodiments. Additional advantages and modifications will be readily apparent to those skilled in the art. The invention in its broader aspects is therefore not limited to the specific details, representative apparatus and methods and illustrative examples shown and described. Accordingly, departures may be made from such details without departing from the scope or spirit of the general inventive concept.

What is claimed is:

1. An implantable closure device for sealing an opening through a biologic tissue membrane against leakage of biological fluid, the closure device comprising:
a fluid sealing plug configured to be positioned at least partially within the opening through a biologic tissue membrane; and
a retainer configured to secure the fluid sealing plug at least partially within the opening, the retainer including:
a unitary and continuous retainer wire structure including:
a proximal retainer wire portion forming a proximal retainer portion configured to be disposed proximally on the biologic tissue membrane,
a distal retainer wire portion forming a distal retainer portion configured to be disposed distally on the biologic tissue membrane, and
a central retainer wire portion forming a central retainer portion connecting the proximal retainer portion and the distal retainer portion;
wherein the central retainer portion is configured to extend through the opening,
the fluid sealing plug is coupled to the central retainer portion,
a generally radial outer surface of the fluid sealing plug is configured to engage a generally radial inner surface of the opening to provide an at least partially sealed interface between the generally radial outer surface of the fluid sealing plug and the generally radial inner surface of the opening,
the retainer is reconfigurable between a low-profile delivery configuration and an implanted configuration,
in the implanted configuration, a diameter of the proximal retainer portion is substantially greater than a diameter of the fluid sealing plug, and
in the implanted configuration, a diameter of the distal retainer portion is substantially greater than a diameter of the fluid sealing plug.

2. The device of claim 1, wherein the fluid sealing plug is coupled to the retainer between the proximal retainer portion and the distal retainer portion.

3. The device of claim 1, wherein the fluid sealing plug is formed of a porous polymer material.

4. The device of claim 3, wherein the fluid sealing plug comprises expanded polytetrafluoroethylene.

5. The device of claim 1, wherein, in the implanted configuration, the diameter of the proximal retainer portion is about 1.25 to about 3.0 times at least one of a diameter of the opening and the diameter of the fluid sealing plug.

6. The device of claim 1, wherein, in the implanted configuration, the diameter of the distal retainer portion is about 1.25 to about 3.0 times at least one of a diameter of the opening and the diameter of the fluid sealing plug.

7. The device of claim 1, wherein the fluid sealing plug has a generally toroidal shape about a longitudinal axis of the closure device.

8. The device of claim 7, wherein the generally toroidal shape of the fluid sealing plug defines a central, axial channel, the channel receiving at least a portion of the central retainer portion therein.

9. The device of claim 1, wherein, in an implanted configuration, the proximal retainer wire portion is generally in the form of a circular loop.

10. The device of claim 1, wherein, in an implanted configuration, the distal retainer wire portion is generally in the form of a circular loop.

11. The device of claim 1, wherein, in an implanted configuration, at least one of the proximal retainer wire portion and the distal retainer wire portion is generally in the form of a polygon.

12. The device of claim 1, wherein, in an implanted configuration, the proximal retainer wire portion is disposed generally in a plane oriented substantially perpendicular to a longitudinal axis of the closure device.

13. The device of claim 1, wherein, in an implanted configuration, the distal retainer wire portion is disposed generally in a plane oriented substantially perpendicular to a longitudinal axis of the closure device.

14. The device of claim 1, wherein the proximal retainer wire portion comprises a first free end of the retainer wire structure and the distal retainer wire portion comprises a second free end of the retainer wire structure.

15. The device of claim 1, wherein, in an implanted configuration, the central retainer wire portion extends generally longitudinally axially between the proximal retainer wire portion and the distal retainer wire portion.

16. The device of claim 1, wherein, in an implanted configuration, the central retainer wire portion extends transversely with respect to a longitudinal axis of the closure device between the proximal retainer wire portion and the distal retainer wire portion.

17. The device of claim 16, wherein, in an implanted configuration, the central retainer wire portion extends substantially obliquely with respect to a longitudinal axis of the closure device between the proximal retainer wire portion and the distal retainer wire portion.

18. The device of claim 1, further comprising at least one of a proximal retainer portion cover at least partially enclosing the proximal retainer wire portion and a distal retainer portion cover at least partially enclosing the distal retainer wire portion.

19. The device of claim 18, wherein the at least one of the proximal retainer portion cover and the distal retainer portion cover comprises a porous polymer material.

20. The device of claim 18, further comprising both of the proximal retainer portion cover and the distal retainer portion cover.

21. The device of claim 1,
wherein the retainer wire structure further comprises a proximal connecting portion between the proximal retainer wire portion and the central retainer wire portion; and
wherein the proximal connecting portion is disposed at a formed angle relative to the proximal retainer wire portion.

22. The device of claim 1,
wherein the retainer wire structure further comprises a distal connecting portion between the distal retainer wire portion and the central retainer wire portion; and
wherein the distal connecting portion is disposed at a formed angle relative to the distal retainer wire portion.

23. The device of claim 1, further comprising a marker configured to be detectable using a medical imaging technique.

24. The device of claim 1, wherein the biologic tissue membrane comprises a meninges and the biological fluid comprises cerebrospinal fluid.

25. A closure device delivery system comprising:
the closure device of claim 1; and
a loading device comprising a generally longitudinal lumen containing the closure device.

26. The system of claim 25, wherein the loading device comprises a pusher assembly configured to deploy the closure device from the loading device.

27. The system of claim 26, wherein the pusher assembly comprises a coupler releasably connected to a free end of the proximal retainer portion.

28. An implantable closure device for sealing an opening through a biologic tissue membrane against leakage of biological fluid, the closure device comprising:
a fluid sealing plug configured to be positioned at least partially within the opening through a biologic tissue membrane, the fluid sealing plug having a diameter; and
a retainer configured to secure the fluid sealing plug at least partially within the opening, the retainer including:
a proximal retainer portion having a proximal retainer portion diameter, wherein the proximal retainer portion is configured to be disposed proximally on the biologic tissue membrane,
a distal retainer portion having a distal retainer portion diameter, wherein the distal retainer portion is configured to be disposed distally on the biologic tissue membrane, and
a central retainer portion configured to extend through the opening and having the fluid sealing plug coupled thereto, the central retainer portion connecting the proximal retainer portion and the distal retainer portion;
wherein the retainer is reconfigurable between an implanted configuration in which the proximal retainer portion diameter is greater than the diameter of the fluid sealing plug and the distal retainer portion diameter is greater than the diameter of the fluid sealing plug, and a delivery configuration in which the distal retainer portion diameter is approximately equal to or less than the diameter of the fluid sealing plug.

29. The device of claim 28, wherein, in the delivery configuration, the proximal retainer portion diameter is approximately equal to or less than the diameter of the fluid sealing plug.

30. The device of claim 28,
wherein the proximal retainer portion at least partially defines a proximal retainer portion central area; and
wherein the proximal retainer portion central area is substantially open.

31. The device of claim 28,
wherein the distal retainer portion at least partially defines a distal retainer portion central area; and
wherein the distal retainer portion central area is substantially open.

32. The device of claim 28, wherein the retainer comprises a retainer wire structure comprising
a proximal retainer wire portion forming the proximal retainer portion,
a distal retainer wire portion forming the distal retainer portion, and
a central retainer wire portion forming the central retainer portion.

33. The device of claim 32, wherein, in an implanted configuration, at least one of the proximal retainer wire portion and the distal retainer wire portion is generally in the form of a circular loop.

34. The device of claim 32, wherein, in an implanted configuration, at least one of the proximal retainer wire portion and the distal retainer wire portion is generally in the form of a polygon.

35. The device of claim 32, wherein the retainer wire structure is constructed from a superelastic metal.

36. The device of claim 28, wherein the biologic tissue membrane comprises a meninges and the biological fluid comprises cerebrospinal fluid.

37. A closure device delivery system comprising:
the closure device of claim 28;
a generally tubular loading device containing the closure device therein, the closure device being in the delivery configuration when contained in the loading device; and
a pusher assembly configured to deploy the closure device from the loading device.

38. The system of claim 37, wherein the closure device is configured to transition from the delivery configuration to the implanted configuration as it is deployed from the loading device.

39. The system of claim 37, wherein the proximal retainer wire portion is releasably coupled to the pusher assembly.

40. The system of claim 37,
further comprising a delivery sheath assembly comprising a lumen extending therethrough;
wherein the loading device and the delivery sheath assembly are configured to releasably couple together such that the lumen of the delivery sheath assembly is generally axially aligned with a lumen of the loading device.

41. The system of claim 40,
further comprising a needle assembly;
wherein the needle assembly and the delivery sheath assembly are configured to releasably couple together such that at least a portion of the needle assembly extends through the lumen of the delivery sheath assembly.

42. A method of closing an opening through a biologic tissue membrane and sealing against leakage of biological fluid, the method comprising:
advancing a generally tubular delivery sheath assembly through an opening through a biologic tissue membrane;
positioning a loading device on the delivery sheath assembly, the loading device comprising a lumen containing a closure device in a delivery configuration;
deploying a distal retainer portion of the closure device distal to a distal surface of the biologic tissue membrane through the opening;
deploying a fluid sealing plug coupled to a central retainer portion of the closure device at least partially into the opening; and
deploying a proximal retainer portion of the closure device proximal to a proximal surface of the biologic tissue membrane;
wherein deploying the distal retainer portion of the closure device, deploying the fluid sealing plug, and deploying the proximal retainer portion comprises deploying the closure device from the loading device and through the delivery sheath assembly.

43. The method of claim 42, wherein deploying the distal retainer portion of the closure device comprises extending the distal retainer portion of the closure device from a distal tip of the delivery sheath assembly and transitioning the distal retainer portion from the delivery configuration to an implanted configuration.

44. The method of claim 42, wherein deploying the proximal retainer portion of the closure device comprises extending the proximal retainer portion of the closure device from a distal tip of the delivery sheath assembly and transitioning the proximal retainer portion from the delivery configuration to an implanted configuration.

45. The method of claim 42, wherein deploying the closure device from the loading device comprises advancing distally a pusher assembly, the pusher assembly being configured to push the closure device distally from the lumen of the loading device and through a lumen of the delivery sheath assembly.

46. The method of claim 45, wherein deploying the proximal retainer portion of the closure device comprises detaching the proximal retainer portion of the closure device from the pusher assembly.

47. The method of claim 46, further comprising, prior to detaching the proximal retainer portion from the pusher assembly, assessing at least one of a position of the closure device and an efficacy of the closure device.

48. The method of claim 42, wherein deploying the fluid sealing plug comprises withdrawing the delivery sheath assembly from the opening.

49. The method of claim 42, wherein advancing the generally tubular delivery sheath assembly through the opening through the biologic tissue membrane comprises advancing together the delivery sheath assembly and a needle assembly extending through the lumen of the delivery sheath assembly at least until a tip of the delivery sheath assembly has penetrated the biologic tissue membrane.

50. The method of claim 49, further comprising, before positioning the loading device on the delivery sheath assembly, removing the needle assembly from the delivery sheath assembly.

51. The method of claim 42, wherein the biologic tissue membrane comprises a meninges and the biological fluid comprises cerebrospinal fluid.
